(19) United States
(12) Patent Application Publication
(10) Pub. No.: US 2013/0289574 A1
(43) Pub. Date: Oct. 31, 2013

(54) DRILLING GUIDE FOR DRILLING COMBIHOLE

(71) Applicant: Padmakar Shinde, Nashik (IN)

(72) Inventor: Padmakar Shinde, Nashik (IN)

(21) Appl. No.: 13/979,061

(22) PCT Filed: Jan. 11, 2012
(86) PCT No.: PCT/IN2012/000031

§ 371 Date: Jul. 10, 2013

(30) Foreign Application Priority Data
Jan. 11, 2011 (IN) 83/MUM/2011

(51) Int. Cl. A61B 17/17

(52) U.S. Cl. CPC A61B 17/1714
USPC 606/96

(57) ABSTRACT

A drilling guide adapted to drill a combihole through a bone for passing ligament grafts for ligament reconstruction, said drill guide comprising: a pair of adjacently located cannulae adapted to drill and form said combihole, said cannulae being conjoined to intersect each other.

### FIELD OF THE INVENTION

This invention relates to the field of biomedical engineering.

Particularly, this invention relates to the field of biomedical engineering related to ligament reconstruction.

Still particularly, this invention relates to a drilling guide for drilling a combihole in a bone.

### BACKGROUND OF THE INVENTION

Knee, in humans, support the entire body weight. It is hence susceptible to injury, apart from wear and tear. The knee is the largest joint in the human body. The knee joint joins the thigh with the leg and consists of two articulations: one between the femur and tibia, and one between the femur and patella. It provides flexion and extension movement apart from slight medial and lateral rotation.

The components of the knee include ligaments; which offer stability by limiting movements. Cruciate ligaments are ligaments which cross each other like the letter ‘X’. Although, they allow a large range of motion, they stabilize the knee. The cruciate ligaments of the knee are the anterior cruciate ligament (ACL) and the posterior cruciate ligament (PCL). The ACL is lateral and the PCL is medial.

The ACL originates from deep within the notch of the distal femur. Its proximal fibers fan out along the medial wall of the lateral femoral condyle. There are two bundles of the ACL—the anteromedial and the posterolateral, named according to where the bundles insert into the tibial plateau. The ACL attaches in front of the intercondyloid eminence of the tibia, being blended with the anterior horn of the lateral meniscus. These attachments allow it to resist anterior translation of the tibia, in relation to the femur.

Anterior cruciate ligament injury is the most common knee ligament injury, especially in athletes. Lateral rotational movements in sports like these are what cause the ACL to strain or tear. Anterior cruciate ligament (ACL) injury or Posterior Cruciate ligament (PCL) is normally treated by reconstruction which nowadays is done or assisted by arthroscopy. The ACL is the most commonly injured ligament of the knee and can be damaged in sports injuries or accidental injuries.

The ACL injury is followed by instability and repeated episodes of giving way which can damage the menisci and result in osteoarthritis or degeneration of the knee if left untreated.

The results of repair have been consistently unsuccessful; hence the ligament is replaced by various autologous grafts like the patellar tendon, hamstrings—Semitendinosus and/or the Gracilis, central third quadriceps or allografts. Recently, the hamstrings are becoming increasingly popular as their harvest does not follow morbidity.

The ACL has two distinct anatomic and functional bundles namely the Anteromedial (AM) bundle and the Posterolateral bundle (PL) named on the basis of their location on the tibia. The AM is the primary restraint against anterior translation of the tibia in flexion and the PL bundle is the primary restraint in extension. The two bundles cross each other in flexion with the AM bundle being posterior to the AL bundle in flexion and moving superior to the PL bundle in extension. In addition the ACL also provides rotational stability.

For reconstruction, two techniques are in use; namely:

(1) Single bundle ACL Reconstruction: where the native ACL is replaced by a quadrupled hamstring graft which is attached to the femur and tibia through single tunnels made in the anatomic centre of the native ACL. This is, by far, the most commonly performed surgery and is simpler to perform than the double bundle technique.

(2) Double bundle ACL Reconstruction: since the native ACL has two different bundles namely the AM and the PL bundles which are taut in different flexion angles a single bundle does not restore the original anatomy of the ACL as well as a double bundle ACL reconstruction.

In this technique, two separate tunnels are drilled into the femur and the tibia in their anatomic centres and two separate grafts are used to recreate the two bundles; so also the two grafts are fixed separately with separate implants in the femur and tibia.

There are reports of residual instability and pivot shift following single bundle reconstruction with early degeneration of the joint. This can be prevented by double bundle reconstruction presumably though no long term studies or evidence is yet available.

In addition to this, the size of the native ACL varies considerably and the size of the graft may not match it in single bundle reconstruction if the native ACL has a very large footprint.

Various implants are in vogue for fixing the soft tissue graft at the femur but can be broadly divided into (1) Suspensory cortical fixation outside the tunnel e.g. the endobutton or the Transfix

(2) Aperture Fixation e.g. with Interference Screw.

It is well settled that the suspensory fixation provides a very strong and secure femoral fixation.

On the tibial side the graft can be secured with interference screw or tied over a suture disc or over a suture post with screw and washer.

The reconstruction requires technically demanding steps like femoral and tibial drilling and in case of Endobutton CL fixation a stepped socket needs to be drilled in the femur without which fixation with endobutton is impossible. Problems like posterior tunnel wall blowout are common and can only be avoided with technical detail.

The PCL is an intracapsular ligament along with the anterior cruciate ligament (ACL) because it lies deep within the knee joint. They are both isolated from the fluid-filled synovial cavity, with the synovial membrane wrapped around them. The PCL gets its name by attaching to the posterior portion of the tibia.

The function of the PCL is to prevent the femur from sliding off the anterior edge of the tibia and to prevent the tibia from displacing posterior to the femur. Common causes of PCL injuries are direct blows to the flexed knee, such as the knee hitting the dashboard in a car accident or falling hard on the knee, both instances displacing the tibia posterior to the femur.

A torn anterior cruciate ligament cannot be “repaired”, and must instead be reconstructed with a tissue graft replacement.

For reconstruction, a hole is drilled through the femur and tibia. The graft forming the ligament is guided through the drill hole and attached in place on the external walls of the bones, typically by endobutton on the femur and sutures tied over a post on the tibia (suspensory fixation). Alternatively the grafts may be secured inside the tunnels at the apertures with bioabsorbable screws or metallic screws (aperture fixation) in order to complete the process of attachment.

Advances in arthroscopy has led to the design and availability of buttons which hold the graft and sit across the drilled hole in the form of an anchor. In the current form of surgery, a hole of a defined diameter is drilled through the medial side of the femur in a transverse direction. After reaching the midpoint of the femur, a narrower tunnel is drilled to complete the hole through to the lateral side of the bone. A button sits as an anchor on this lateral side, atop the cavity defined by the hole.

However, it has been observed that the anchor buttons available in the market work on the pre-condition that the hole is accurately drilled in accordance with specified parameters of dimensions.

Depending upon the numerous kinds of cases and bone structure and size, it becomes difficult for the surgeon to drill actual textbook holes, in spite of precision equipment. It has been observed that ‘blowouts’ may occur, rendering hole diameters larger that the length of the anchor buttons available to the surgeon.

In cases of double tunnel reconstruction, the Double Bundle PCL Guides give versatility in creating appropriate socket placement using anatomical constants or directly visualizing the intended socket diameter with the guides. Two holes side by side form the double tunnel to receive the double bundle.

The grafts are passed through respective tunnels for securing.

A drill guide is used for drilling a hole through a bone.

There is a need for a drill guide for drilling a combihole or a dual hole through a bone.

### OBJECTS OF THE INVENTION

An object of the invention is to provide drilling tools to drill tunnel, combiholes, dual holes, or the like through a bone.

### SUMMARY OF THE INVENTION

For the purposes of this invention, a ‘button’ relates to an anchorage device adapted to provide anchor support to a ligament graft. Typically, the button sits across the cavity of a hole through which the graft is passed. The button body has pre-defined dimensions so that the button is adapted to sit across a cavity in a bone.

This invention also discloses a new bicortical combihole technique of double bundle ACL or PCL reconstruction wherein there is no socket bicortical tunnel single bundle ACL or PCL reconstruction.

According to this invention, there are provided instruments for providing a novel Bicortical “Combihole”, through the bone, for anatomic double bundle ACL or PCL reconstruction.

According to another aspect of this invention, there is provided a drilling guide adapted to drill a tunnel through a bone for passing ligament grafts for ligament reconstruction. The drill guide, according to this invention, includes a pair of adjacently located cannulae which is adapted to drill a combihole. A combihole is a hole formed by two adjacently partially overlapping holes. The confluence of two tunnels is labelled combihole because it resembles the shape of the screw holes in a locking plate with combihole for locking or dynamic hole options.

In accordance with another embodiment of the drilling guide, it may include a sharp bevel in between the two drill cannulae to help in removing the bridge of remaining bone in between the two tunnels of the combihole to allow smooth passage of the Bridge button DT.

According to this invention, there is provided a drilling guide adapted to drill a combihole through a bone for passing ligament grafts for ligament reconstruction, said drill guide comprises: a pair of adjacently located cannulae adapted to drill and form said combihole, said cannulae being conjoined to intersect each other.

According to this invention, there is also provided a drilling guide adapted to drill a combihole through a bone for passing ligament grafts for ligament reconstruction, said drill guide comprises: a pair of adjacently located cannulae adapted to drill and form said combihole, said cannulae being spaced apart by a sharp bevel in order to remove the bridge of remaining bone in between the two tunnels of the combihole formed by said cannulae respective to allow smooth passage of a button element.

Typically, each of said cannulae is an elongate cylindrical shaft.

Typically, said sharp bevel is a metal element with sloping edges which confluence to make a sharp edge at the distal end of said guide.

Preferably, said bevel breadth is a 3.1 mm bridge.

Additionally, said guide includes markings on both (front and back) sides or on all 4 sides to measure the tunnel that it drills.

### BRIEF DESCRIPTION OF THE ACCOMPANYING DRAWING

The invention will now be described in relation to the accompanying drawings, in which:

FIGS. 1a and 1b illustrate a schematic of the lateral condyle of the femur bone with AM tunnel and PL tunnel according to different surgical procedures;

FIG. 2 illustrates a schematic of the AM tunnel and PL tunnel in the tibia;

FIGS. 3a, 3b, and 3c illustrate a schematic of various AM portals and PL portals in the tibia

FIG. 4 illustrates a schematic of the button used for double bundle reconstruction surgery with the sutures therein;

FIG. 5 illustrates a schematic of the button used for double bundle reconstruction surgery with the sutures therein and a spacer element in between the bundles;

FIGS. 6, 7, 8, 9, and 10 illustrate various combinations of holes of the tunnel that are drilled for surgery;

FIGS. 11, 12, and 13a illustrate a schematic of the button used for single bundle reconstruction surgery and in case of lateral cortex blowout;

FIG. 13b illustrates a schematic of the button used for double button reconstruction surgery and in case of lateral cortex blowout;

FIG. 14a illustrates a schematic of a drill guide being inserted into the bone to drill two holes;

FIG. 14b illustrates a cross-section of the hole drilled by the drill guide of FIG. 15a;

FIG. 15b illustrates a drill a guide according to this invention;

FIG. 15a illustrates a close-up view of the drill guide of FIG. 16a;

### DETAILED DESCRIPTION OF THE ACCOMPANYING DRAWINGS

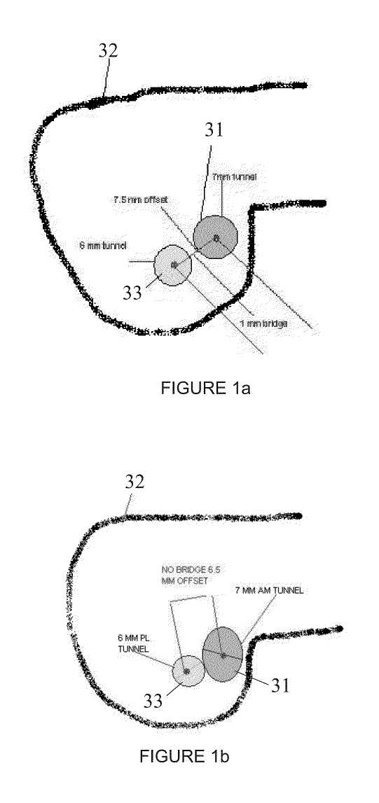

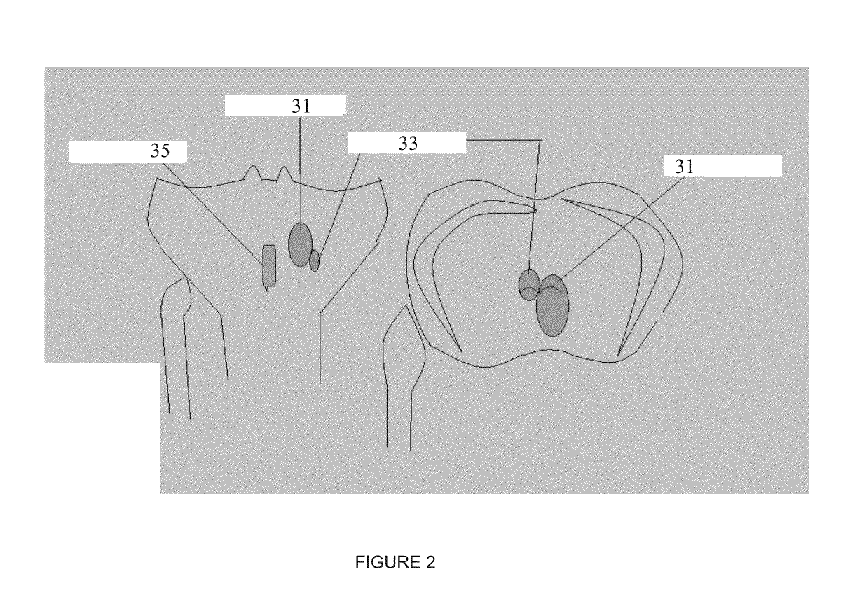

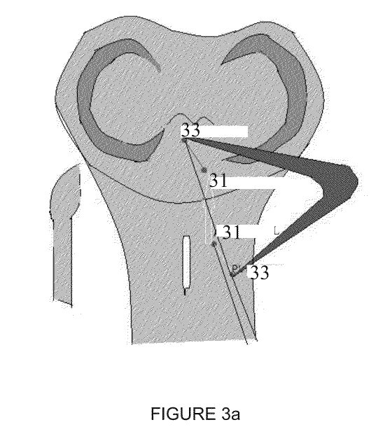

FIGS. 1a and 1b illustrate a schematic of the lateral condyle (32) of a femur bone with AM tunnel (31) and PL tunnel (33) according to different surgical procedures. FIG. 2 illustrates a schematic of the AM tunnel (31) and PL tunnel (33) in the tibia. Reference numeral 35 refers to tuberal tuberosity. FIGS. 3a, 3b, and 3c illustrate a schematic of various AM tunnel and PL tunnel in the tibia.

A button (100) is formed by an elongate body of pre-defined dimensions. The button body has pre-defined dimensions so that the button is adapted to sit across a cavity in a bone. This button is the subject matter of a co-pending patent application with patent application number 83/MUM/2011 and has a bridge portion illustrated by reference numeral 12.

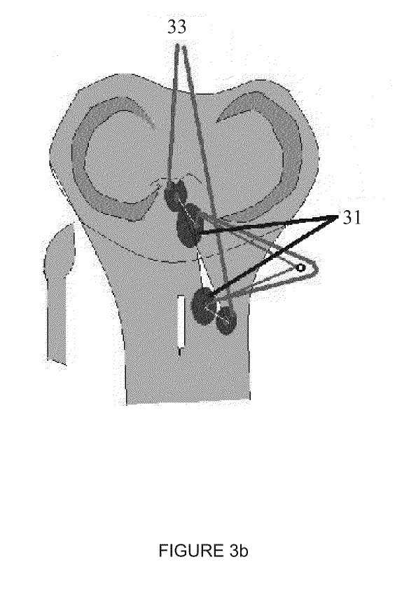

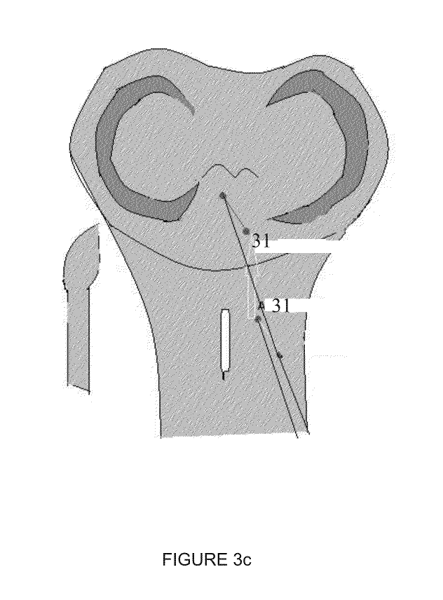

FIG. 4 illustrates a schematic of the button used for double bundle reconstruction surgery with the suture loops therein. FIG. 5 illustrates a schematic of the button used for double bundle reconstruction surgery with the sutures therein and a spacer element (40) in between the bundles (42, 44). Reference numeral 42 refers to AM bundle. Reference numeral 44 refers to PL bundle. Reference numeral 41 refers to pulling sutures. Reference numeral 43 refers to flipping sutures. Hole (45) for interference screw thread is centrally located.

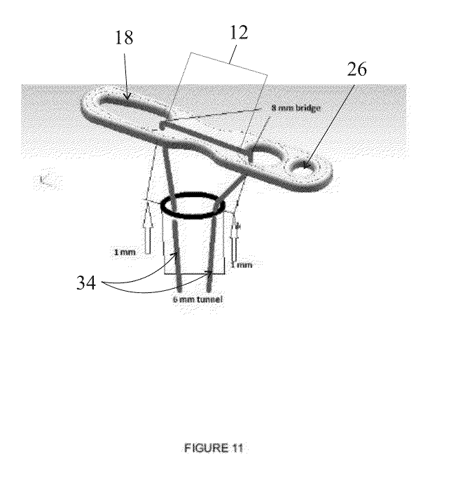

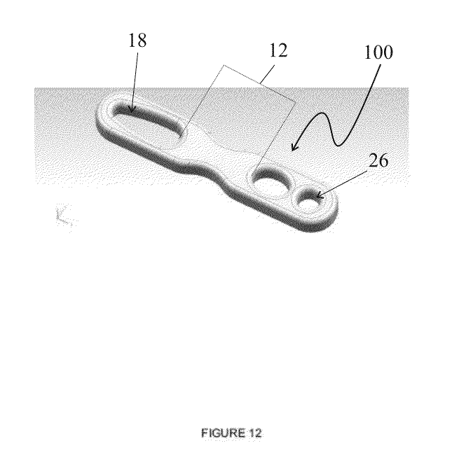

FIGS. 11, 12, and 13 illustrate a schematic of the button used for single bundle reconstruction surgery and in case of lateral cortex blowout.

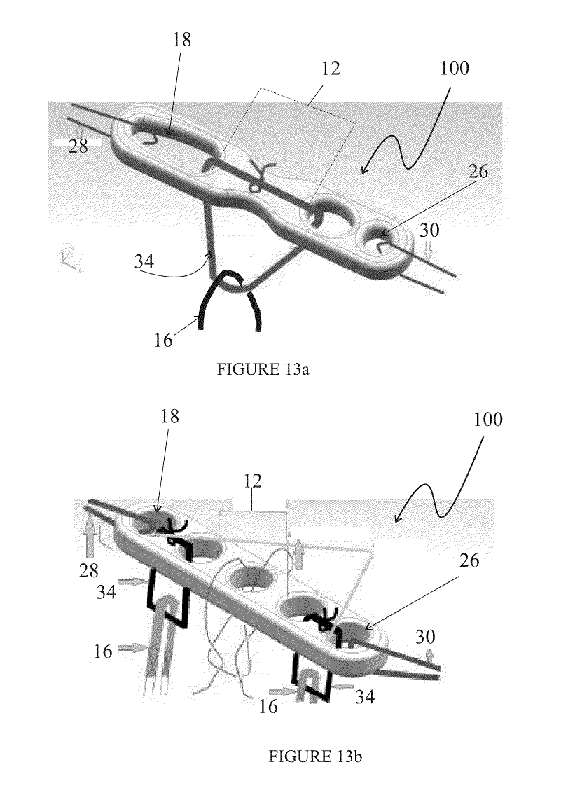

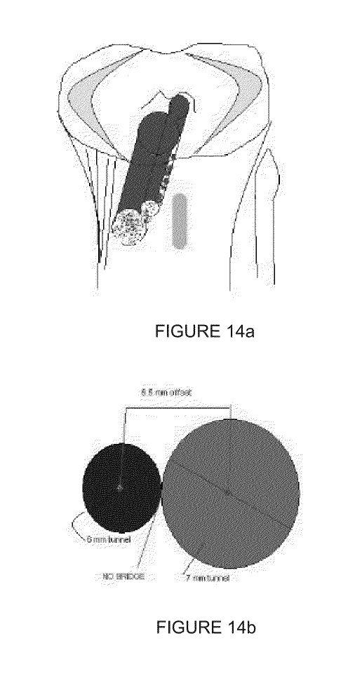

FIG. 13b illustrates a schematic of the button used for double button reconstruction surgery and in case of lateral cortex blowout.

A first laterally located hole (18) at a first longitudinal edge of the button may be used for passing sutures which act as the pulling suture (28). This pulling suture aids in pulling the button along with graft through a formed tunnel. Typically, the button is passed through the tunnel in an operative vertical position along its longitudinal axis.

A second laterally located hole (26) at a second longitudinal edge (longitudinal diametrically opposite to said first longitudinal edge) of the button may be used for passing sutured which act as the flipping suture (30). This flipping suture is typically engaged, by the surgeon, once the button has been pulled out of the tunnel on the lateral side of the bone. It aids in flipping (configuring) button from its operative vertical position (for pulling purposes) to its operative horizontal position wherein it can rest upon the cavity, transversely.

Ideally, the bridge element sits across the cavity with the lateral exterior-most holes placed outside the cavity and resting upon the solid portion of the bone.

In a preferred embodiment and method, the bridge is marginally lesser than the cavity. The margin which is the differential incorporates the diameter of the sutures on either end. Thus the sutures pass through the inner edge of the holes and are plugged in between said inner edge of the hole and the wall of the tunnel. This provides a secure fit of the button onto the cavity.

In a preferred embodiment, the holes are placed on either side of said solid bridge element whereby providing the bridge formed by the solid element for providing a salvaging component in lateral femoral cortex blowout conditions in ACL or PCL reconstruction.

There is provided a technique of Bridge Button in the novel bicortical tunnel “no socket” ACL or PCL reconstruction surgery.

In a single bundle loop technique, two holes, equidistant from the centre, on either side, are used to pass a suture loop (34) which provides a support loop for the ligament graft. (as shown in FIGS. 11 and 13)

In a double loop technique, two pairs of holes, equidistant respectively from the centre, on either side, are used to pass a suture loop (36, 38) on either side which provides a pair of support loops for a pair of ligament grafts respectively. (as shown in FIGS. 4 and 5)

According to this invention, there are also provided instruments for providing a novel Bicortical “Combihole”, through the bone, for anatomic double bundle ACL or PCL reconstruction.

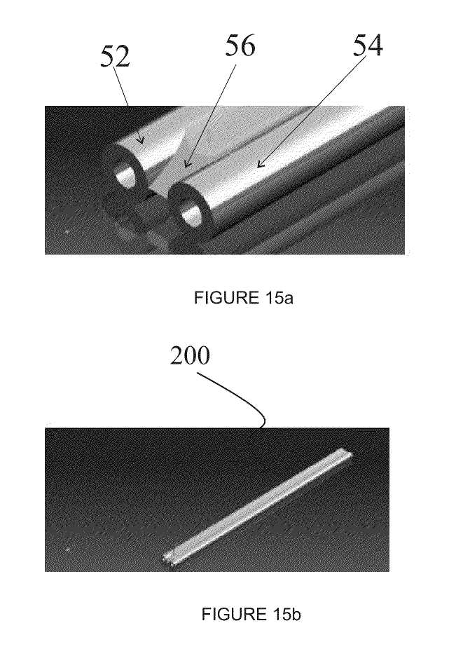

FIG. 15a illustrates a schematic of a drill guide being inserted into the bone to drill two holes; and FIG. 15b illustrates a cross-section of the hole drilled by the drill guide of FIG. 15a.

The implant provides strong cortical fixation at both the femoral and tibial ends, separates the graft into two distinct bundles, allows individual tensioning and fixation of the two bundles in different flexion angles and can be combined with aperture fixation like bioscrew or the special insert or cage.

The “bridge” principle, which defines the incorporation of a bridge, basically relies on the distance or the “Bridge” provided in between the holes of the implant or in between the two strands of a single loop or in between the two strands of two different loops of sutures. The sutures may be ethibond suture, mersilene tape, polyester or any suitable strong material.

This bridge is of critical importance to effectively space the suture loop inside the blowout tunnel; e.g. in a 10 mm cortical blowout, an 8 mm bridge with the suture loops can effectively fill the tunnel internally by the suture loops leaving no space for side to side movement of the implant and effects rigid, secure, reliable and reproducible femoral cortical fixation. This also eliminates the risk of dislodgement or loss of fixation completely.

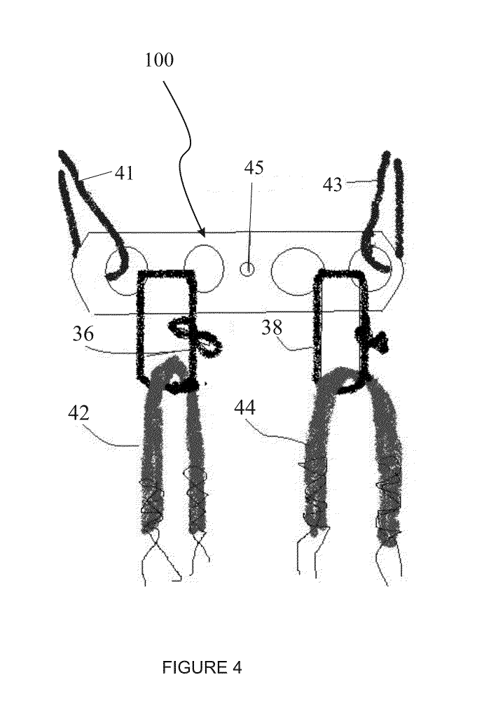

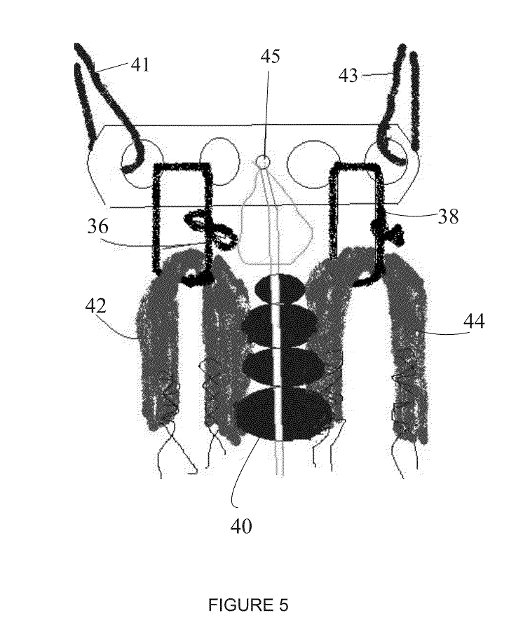

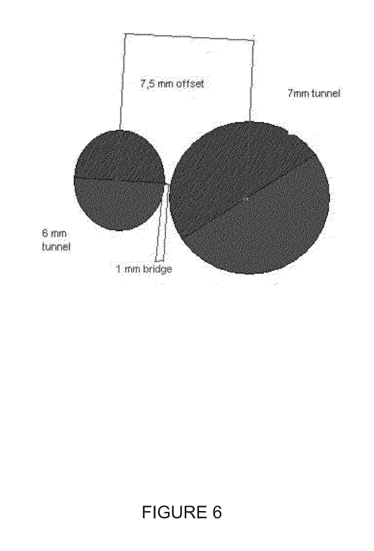

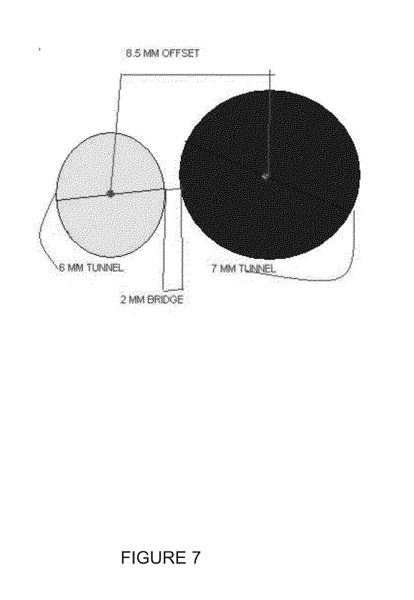

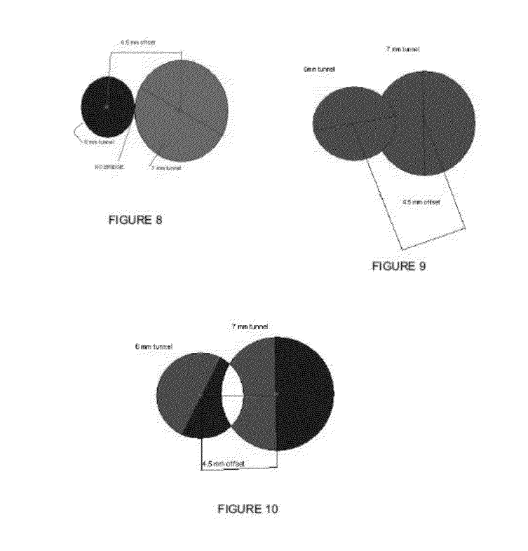

FIGS. 6, 7, 8, 9, and 10 illustrate various combinations of holes of the tunnel that are drilled for surgery.

According to another embodiment of this invention, there is provided a special depth gauge with markings on both (front and back) sides or on all 4 sides to measure the femoral tunnel.

An implant designed for use in lateral cortex blowout during ACL or PCL reconstruction surgery.

According to this invention, there is provided a drilling guide (200) adapted to drill a tunnel through a bone for passing ligament grafts for ligament reconstruction. The drill guide, according to this invention, includes a pair of adjacently located cannulae (52, 54) which is adapted to drill a combihole. A combihole is a hole formed by two adjacently partially overlapping holes. This combihole is, typically, a figure of 8 shaped hole. The confluence of two tunnels is labelled combihole because it resembles the shape of the screw holes in a locking plate with combihole for locking or dynamic hole options.

FIG. 15b illustrates a drill a guide according to this invention.

FIG. 15a illustrates a close-up view of the drill guide of FIG. 15b.

Typically, a set of offset drill guides is designed ranging from 5 mm offset to 10 mm offstet with 0.5 mm increments for creating a combihole tunnel in the tibia as well as the femur to allow housing the two bundles of the ACL or PCL in the two tunnels and to allow passage of the Bridge button DT through the intervening space for secure fixation on the lateral femoral cortex.

In accordance with another embodiment of the drilling guide, it may include a sharp bevel (56) in between the two drill cannulae to help in removing the bridge of remaining bone in between the two tunnels of the combihole to allow smooth passage of the Bridge button DT.

Preferably, a 3.1 mm bridge between the two drill hole guide cannulae ensures enough bone removal for the passage of a 2 mm thick Bridge button DT.

A variety of tunnel diameters and their combinations are possible for the varying graft diameter sizes and the variance in the dimensions in the footprint of the native ACL. With the proper offset, any kind of two tunnel diameters can be matched to create a combihole.

The offset drill guides offers an option of creating two tunnels with following 3 exemplary non-limiting variations:
1. Two tunnels with 1 or 2 mm bridge: e.g. for graft diameters of 6 and 7 mm respectively for the PL and AM bundle, in case bridge of bone of 2 mm is desired, an offset of 6.5 (sum of radius of the two tunnels)+2 mm=8.5 will be required. If a bridge is preferred between the two tunnels, the intervening bridge must be removed with small chisels or small box rongeurs or the sharp bevel provided in the offset drill guide in claim 15 to allow passage of the Bridge Button DT. Such a bridge would be recommended only if the native ACL has a very large footprint and the graft diameter is relatively small. This intervening bridge may pose some difficulty in the passage of the implant and the graft but can be managed by broadening the bridge or increasing the tunnel diameters.
2. Two tunnels without any bridge or zero bridge: e.g. for graft diameters of 6 and 7 mm respectively for the PL and AM bundle an offset of 6.5 (sum of radius of the two tunnels) is required. In most instances an offset with zero bridge between the tunnels is recommended. In practice this “no bridge” tunnels get automatically merged with blowout of the intervening wall and also retain distinctness of the two tunnels. The intervening space allows sufficient space for the Bridge button DT to pass without much difficulty.
3. Two tunnels with varying amount of overlap of the two tunnels creating a single blowout tunnel: e.g. for graft diameters of 6 and 7 mm respectively for the PL and AM bundle an offset of 6.5 (sum of radius of the two tunnels) minus the desired 1 or 2 mm overlap=6.5-2=4.5 mm offset is required.

If offset less than 6.5 is selected, the two tunnels will merge creating a blowout tunnel. The distinctness of the two separate tunnels can be lost with this blowout tunnel and one will have to rely on interference screw fixation to recreate the two bundle separation. Such tunnel might be required in small knees or where the native ACL footprint is very small.

During surgery, the PL tunnel (or the AM tunnel) on the lateral femoral condyle is marked at its anatomic insertion and a guide wire is passed in its anatomic centre with the knee in 110 degrees of flexion; care is taken to direct the wire away from the insertion of the lateral collateral ligament.

The appropriate offset (e.g. a 6.5 mm offset drill guide for 6 mm PL bundle and 7 mm AM bundle for no bridge tunnels) drill guide is passed over this guide wire and positioned over the second AM tunnel

The offset to be used is decided on the basis of the diameter of the native ACL footprint and the graft diameters available. Another guide wire is passed through this anatomic AM site. The wire exit point is directed away from the anatomic LCL site to avoid injury to the lateral collateral ligament. The knee is flexed to 110 degrees to avoid injuring lateral knee structures during tunnel drilling.

Both the tunnels are drilled throughout the entire length of the tunnel creating a bicortical hole and not a stepped hole unlike the conventional manner, to about 1 mm less size and then dilated to the desired diameter.

Any intervening bridge of bone between the tunnels is removed with the sharp bevelled edge of the drill guide or with ordinary small arthroscopic osteotomes or chisels.

For Graft-Loop-Button preparation, the graft is prepared in a manner to allow optimum diameters for either tunnel; the bigger one being used for the AM tunnel.

The tunnel lengths are measured and appropriate sized loops are made to allow at least 20 mm of graft insert in each tunnel. The grafts are draped over the two loops separately and markings with marker pen are made at 20 mm and 35 mm for graft insert and flipping distance respectively. One of the bundles is marked with different colour to help distinguish between the two bundles.

Strong passing suture or fibre wire is passed through one end hole and preferably a different colour strong suture for flipping is passed through the end hole on the other side

With a continuous loop technology, the appropriate sized loop can be selected without the need for manual knotting. With a self-locking suture technology there is no need to calculate the loop lengths. In addition, the loops can be left longer for easy passage of the Bridge button which once locked individual bundles can be pulled inside the tunnels separately making graft passage simpler and easier.

The AM tunnel is marked on the tibial anatomical insertion site with a guide pin using conventional ACL jig and the wire is passed into the joint. Then the Offset drill guide is passed onto this guide wire and a mark is made on the medial tibia for the second PL tunnel protecting the MCL.

A special ACL jig is passed into the joint which has markings on the aimer in millimeters to adjust the distance of the aimer exactly to the same distance as the offset drill guide used e.g. 6.5mm. This jig is placed in the anatomic centre of the PL bundle intraarticularly at 6.5 mm distance from the previous guide wire and its ratcheted wire guide placed on the mark made on the tibia with the offset drill guide so that both the internal and external tunnel centres are exactly placed at the desired offset to create a combihole.

The second guide wire is then passed to emerge through the anatomic centre of the other bundle.

Both tunnels are drilled to 1 mm less than the graft diameter; the tunnels are dilated to the desired diameter with serial dilators. Any bridge of intervening bone is removed with the offset drill guide gouge.

Alternatively, the tunnels may be drilled with a coring reamer to recover the bone grafts for using in the cage for the intratunnel implant.

A passing suture is passed retrograde through the femoral tunnel into the tibial tunnel to retrieve the pulling and flipping sutures out through the lateral femoral cortex. The pulling sutures pull the Bridge Button DT and the graft through the tibial and femoral tunnels out through the lateral femoral cortex. The button is flipped and locked in position.

The suture of the central hole is retrieved through the AM portal and an interference screw is passed over it to provide aperture fixation. The suture may be used to tie knots to secure the screw to the Bridge button so that in case of inadvertent loosening it does not get dislodged into the joint.

The technical advancement lies in the fact that a combihole can be effectively drilled using this drilling guide. It is useful for outside in tunnel drilling techniques. It is useful in children where growth plate avoidance may dictate outside in drilling. Single member assembly avoids the possibility of disengagement.

While considerable emphasis has been placed herein on the particular features of this invention, it will be appreciated that various modifications can be made, and that many changes can be made in the preferred embodiments without departing from the principles of the invention. These and other modifications in the nature of the invention or the preferred embodiments will be apparent to those skilled in the art from the disclosure herein, whereby it is to be distinctly understood that the foregoing descriptive matter is to be interpreted merely as illustrative of the invention and not as a limitation.

1. A drilling guide adapted to drill a combihole through a bone for passing ligament grafts for ligament reconstruction, said drill guide comprising: a pair of adjacently located cannulae adapted to drill and form said combihole, said cannulae being conjoined to intersect each other.

2. A guide as claimed in claim 1 wherein, each of said cannulae is an elongate cylindrical shaft.

3. A guide as claimed in claim 1 wherein, said guide includes markings on both (front and back) sides or on all 4 sides to measure the tunnel that it drills.

4. A drilling guide adapted to drill a combihole through a bone for passing ligament grafts for ligament reconstruction, said drill guide comprising: a pair of adjacently located cannulae adapted to drill and form said combihole, said cannulae being spaced apart by a sharp bevel in order to remove the bridge of remaining bone in between the two tunnels of the combihole formed by said cannulae respective to allow smooth passage of a button element.

5. A guide as claimed in claim 4 wherein, each of said cannulae is an elongate cylindrical shaft.

6. A guide as claimed in claim 4 wherein, said sharp bevel is a metal element with sloping edges which confluence to make a sharp edge at the distal end of said guide.

7. A guide as claimed in claim 4 wherein, said bevel breadth is a 3.1 mm bridge.

8. A guide as claimed in claim 4 wherein, said guide includes markings on both (front and back) sides or on all 4 sides to measure the tunnel that it drills.
